(19) United States
(12) Patent Application Publication
(10) Pub. No.: US 2009/0022623 A1
(43) Pub. Date: Jan. 22, 2009

(54) Peptides Capable of Functioning as Mimotopes for Hormonal Analytes

(75) Inventors: Robert A. Badley, Bedford (GB); Mark J. Berry, Bedford (GB); Samantha C. Williams, County Dubline (IE)

Correspondence Address: FOLEY HOAG, LLP;PATENT GROUP, WORLD TRADE CENTER WEST, 155 SEAPORT BLVD, BOSTON, MA, 02110, US

(73) Assignee: Inverness Medical Switzerland GmbH, Zug (CH)

(21) Appl. No.: 11/779,009

(22) Filed: Jul. 17, 2007

Related U.S. Application Data
(63) Continuation of application No. 09/920,306, filed on Aug. 2, 2001, now abandoned.

(30) Foreign Application Priority Data
Aug. 3, 2000 (EP) 00306613.1

(51) Int. Cl. G01N 31/22; C07K 7/00; C07H 21/04

(52) U.S. Cl. 422/57; 530/330; 530/329; 530/328; 536/23.1

(57) ABSTRACT

A purified peptide mimotope which is capable of binding specifically to an antibody specific to estradiol. Also disclosed is an immunoassay test device for the detection in a sample of estradiol, the immunoassay test device comprising a peptide mimotope of estradiol, and an antibody capable of binding specifically to the peptide mimotope to generate a detectable signal.

### FIELD OF THE INVENTION

This invention relates to the discovery that certain peptide molecules have similar reactive properties as certain steroidal compounds, notwithstanding the significant structural dissimilarities between such compounds, and are thus capable of functioning as mimotopes of the steroidal compounds in, for example, displacement immunoassays designed for the detection of steroids.

### BACKGROUND OF THE INVENTION

As a matter of general definition, an epitope is that region of a particular antigen which contains the critical binding region of the antigen necessary for triggering an immunity-related antibody binding response. Epitopes are also often referred to in the alternative as antigenic determinants.

Understanding the structures of epitopes as well as their specific binding reactions to particular antibodies is of significant interest to many, as such an understanding could lay the foundation for advancements in the pharmaceutical, diagnostic and health industries. To facilitate this understanding, in recent years academic institutions and industry have constructed what are termed epitope libraries.

Epitope libraries are large collections of variable amino acid sequences that are displayed, for example, on the surfaces of bacteriophage. Each sequence corresponds to a particular epitope of a particular antigen. Often, the epitope libraries will consist of many millions of these short amino acid sequences, sometimes even as many as one hundred million sequences or more. Representative epitope libraries are described in detail in Luzzago A., et al. Mimicking of discontinuous epitopes by phage-displayed peptides, I. Epitope mapping of human H ferritin using a phage library of constrained peptides. Gene 128, 51-57 (1993).

Once an epitope library is constructed, antibodies or other binding proteins can be utilised to select specifically for a particular epitope. The epitope can then be sequenced, either directly or by first identifying the corresponding DNA sequence and then by transcribing and translating that DNA sequence into the corresponding amino acid sequence. By such techniques, the binding regions of antigenic compounds and molecules can be determined; and it can be readily envisioned that once the binding regions of particular antigens are known, powerful biotechnology applications—such as the design of vaccines using particular epitopes—can be achieved.

Despite the apparent power of epitope library screening techniques, they have heretofore been used primarily only to identify and sequence the specific binding regions of particular antigens. This in and of itself serves to limit the type and scope of biotechnology applications that can be based on this technology.

In Scott, Discovering Peptide Ligands Using Epitope Libraries, Trends in Biochemical Science 17, pp. 241-245 (July 1992), an extension of conventional epitope library techniques is disclosed. Specifically, Scott asserts that epitope libraries can be used to identify and map peptide mimotopes for known antigens. A mimotope is a molecular sequence which “mimics” the epitopic region of a particular antigen, but which does not contain the specific amino acid sequence which comprises the epitope. Thus, a mimotope is structurally distinct from an epitope, though functionally it is very similar as it is capable of binding in a similar fashion to the binding cleft of the antibody directed to the antigen containing the particular epitope.

Though mimotopes technically can be any molecules or sequence of molecules which mimic an epitope, they are most often small, low molecular weight peptides which comprise short sequences of amino acids. Because they are most typically small peptides, they have been thought to be constrained in what they can mimic. Specifically, it has been a generally held belief that peptide mimotopes could be identified for numerous protein based antigens (i.e. those antigens with peptide epitopes), but that because of the complex structure of antibody binding clefts, and the correspondingly complex nature of the antibody binding response, all of which it was believed would require a close similarity in structure between the mimotope and epitope in order for mimotope-antibody binding to occur, the identification of peptide mimotopes for non-protein based antigens would be difficult to achieve. In general this belief has been proved correct, although there are a few isolated exceptions.

For example, in Random peptide libraries: A source of specific binding molecules: Devlin J J: Science 249, 404-406 (1990), peptide mimotopes have been identified for biotin, an essential vitamin necessary for certain enzymatic carboxylation reactions in living cells. Though biotin is not peptidal in structure, it is nevertheless similar in size and structure to several amino acids (for example, histidine). Thus, it was not unexpected that a peptide mimotope for such a molecule could be identified.

Similarly, in Peptide ligands for a sugar binding protein isolated from a random peptide library: Oldenburg, K R et al., Proc. Nat. Acad. Sci. USA, 89, 5393-5397 (1992), peptide mimotopes for the mannopyranoside ligand of concanavalin A have been identified. Again though, such mimotopes are of similar size and have a similar structural configuration to the epitopes which they mimic and thus their identification was less than surprising. Mimotopes of certain forms of DNA have also been identified, as described in Sibille et al., Mimotopes of polyreactive anti-DNA antibodies identified using phage displayed peptide libraries: Eur J Immunol., 27, 1221-1228 (1997)

Notwithstanding these few limited discoveries, the identification of peptide mimotopes from epitope libraries (or other means) for complex non-protein molecules such as steroidal compounds has not occurred. Furthermore, due to the clear structural differences between such peptide mimotopes and complex non-protein molecules such as steroids, the mere existence of these mimotopes has heretofore been questioned.

WO 96/16322 discloses an affinity-based process for recovering specific binding agents with high affinity for a particular target ligand, which involves the use of first and second analogues of the target ligand. Page 4 of the document mentions that one of the analogues used in the process may be an epitope mimic, “i.e. a small molecule, generally of synthetic origin, such as a short peptide, which behaves in a manner comparable to the binding site (epitope) of the target ligand”.

Although the document includes examples of the process in which the target ligand is a steroid (specifically, estrone-3-glucuronide, “E3G”) none of these examples involve the use of a peptide mimotope of a steroid, and there is no disclosure of a specific example of, nor any experimental evidence relating to, a peptide mimotope of a steroid. Indeed at page 4, immediately following the passage quoted above, WO 96/16322 states “Especially when the target ligand is E3G, said first analogue can be estrone. Preferably said second analogue is estriol glucuronide. Alternatively, estradiol-3-glucuronide can be used as the second analogue; in this case, estriol-3-glucuronide may optionally be used as the first analogue”.

Thus, whilst WO 96/16322 refers to the use of peptide epitope mimics of target ligands, and also refers to steroid target ligands, there is no explicit disclosure or suggestion of a peptide mimic for a steroid target ligand. Indeed the only mention of particular analogues for steroid target ligands are other, closely-related, steroids. Accordingly, the person skilled in the art would not deduce from the content of WO 96/16322 that peptide mimics for steroid analogues actually existed or could be made, and would not have any reasonable expectation of success in this regard as there is no evidence to suggest that such existed.

A brief reference, along similar lines to that discussed above, appears in WO 99/27356 (page 10) but again without specific examples or any experimental evidence.

Saviranta et al, (1998 Bioconjugate Chem. 9, 725-735) disclose an assay for estradiol using Fab fragments specific for estradiol. There is no mention or suggestion of a peptide mimotope of estradiol which binds to the Fab fragments.

Slootstra et al, (1997 Journal of Molecular Recognition 10, 217-224) describe a screening method to identify synthetic peptides that mimic epitopes, but those authors only ever refer to mimics of protein or peptide antigens, and there is no recognition or suggestion that peptide mimotopes might be available in respect of steroid compounds.

Lastly, U.S. Pat. No. 5,635,182 (McCoy & Lu) relates to subject matter very different to the present invention and is generally concerned with DNA sequences encoding thioredoxin-like polypeptides. There is a brief disclosure (Seq. ID No. 2 therein) of a 20mer peptide which includes the tripeptide sequence Phe-Glu-Asp.

### SUMMARY OF THE INVENTION

This invention is based on the unexpected discovery that despite significant structural differences, peptide mimotopes for certain steroidal compounds do in fact exist and can be advantageously used, for example, in competitive or displacement-type immunoassays designed for the detection of steroids in a sample. In this regard, the present invention is directed to a purified peptide mimotope which is capable of binding specifically to an antibody specific to estradiol, and to isolated nucleic acid sequences encoding the purified peptide mimotope. It is also directed to an immunoassay test device for the detection in a sample of estradiol, the immunoassay comprising the peptide mimotope, as well as an antibody capable of binding specifically to the peptide mimotope to generate a detectable signal.

The present invention provides numerous advantages. In addition to the peptides being capable of being utilised as immunogens, the peptide mimotopes can be used to construct new, or improve the performance of old, immunoassay test formats and devices. They can, for example, be utilised essentially to “tune” the signal in conventional displacement assays for the detection of estradiol. Further, they can be bound directly to certain assay surfaces which are otherwise non-compatible with estradiol, the estradiol on such surfaces needing to be bound to the surface by complexing with another—often proteinaceous—molecule. Other advantages will become readily apparent in the description of the invention below.

### DETAILED DESCRIPTION OF THE INVENTION

The peptide mimotopes of the invention are capable of specific binding to any antibody which is specific to estradiol. Estradiol as used herein shall be taken to mean estradiol or metabolites thereof (e.g. the preferred estrone-3-glucuronide), as well as any related steroidal compounds having a basic estrone structure. Such related compounds are exemplified by, but not necessarily limited to, estriol, 16-epiestriol, 17-epiestriol, 17-β-estradiol 3-(β-D-glucuronide), estriol 3-(β-D-glucuronide), estrone, 17 α-ethynylestradiol, and 16 α-hydroxyestrone.

By specific binding it is meant that the mimotope is capable of being bound to the antigen-binding site of an antibody in a selective fashion in the presence of excess quantities of other materials not of interest, and tightly enough (i.e. with high enough affinity) that when used in an immunoassay, it provides a useful assay result. Similarly, an antibody “specific to estradiol” is one which is capable of binding to estradiol (or related compounds) in a selective fashion in the presence of excess quantities of other materials not of interest, and tightly enough that when used in an immunoassay it provides a useful assay result.

The antibody to which the peptide mimotopes are capable of being specifically bound can be any antibody, fragment or construct thereof, having a binding specificity for estradiol or metabolites thereof. Various forms of such antibodies are contemplated which may include monoclonal or polyclonal antibodies, Fv, Fab, ScFv and the like. Also contemplated are multivalent and/or multispecific constructions which have been described in the literature and comprise two or more polypeptide chains—see for example, patent application Harris et al., WO 94/09131 and Davis et al., WO 97/14719—or are based on a ‘double ScFv’ approach, wherein the multivalency arises when two or more monovalent ScFv molecules are linked together, providing a single chain molecule comprising at least four variable domains, as described, for example, in Whitlow et al., WO 93/11161 and Mezes et al., WO 94/13806.

The antibodies, when utilized with the peptide mimotopes in an immunoassay test device, can be constructed by methods known in the art. Techniques such as those exemplified in Verhoeyen and Windust, Advances in Antibody Engineering in Molecular Immunology: Frontiers in Molecular Biology, 2nd Ed., published by Oxford University Press, pp. 283-325 (Oxford, 1995) and Price et al. Principles and Practice of Immunoassays, 2nd Ed., published by Macmillan Publishers Ltd (London, 1997) are suitable. Many antibodies may also be obtained commercially. For the estradiol metabolite, estrone-3-glucuronide, a monoclonal antibody is described in Linscott's Directory of Immunological and Biological Reagents (10^{th} edition 1998-9) and may be obtained from OEM Concepts Inc, Toms River, N.J., USA.

As described in the accompanying examples, the peptide mimotopes of the invention have been identified from epitope libraries by various screening techniques. They have also been identified from peptide libraries constructed from the known naturally occurring amino acids.

The peptide mimotopes will contain a minimum core binding region. That is, they will include a minimum continuous amino acid sequence which is necessary for imparting to the mimotope the capability of specific binding to the target antibody. Preferably, the region is such that the affinity of the mimotope for the binding reaction to a single antibody binding site in solution is greater than or equal to 10⁵ L/mole. Methods of determining binding affinity are routine for those skilled in the art, and the binding affinity of a particular mimotope for a particular antibody can be readily measured with the benefit of the techniques described herein and using the common general knowledge of those skilled in the art.

The peptide mimotopes can be any size, though it is preferable that they be smaller than that which would allow for tertiary or globular structuring to occur. Thus, they are typically no larger than 30, and preferably no greater than 20, amino acids in length. The core binding region of each mimotope will typically be less than 12 amino acids, preferably less than 7 amino acids, and optimally between 3 to 6 amino acids in length. Preferred mimotopes are identified in the examples below.

In particular, the inventors have found that the core binding region of many mimotopes in accordance with the invention comprises one of the three tripeptide sequences identified as follows: Xaa-Glu-Asp; Phe-Xaa-Asp; and Phe-Glu-Xaa. Thus, in general, preferred mimotopes will comprise one of these three tripeptide sequences (typically, Phe-Glu-Asp), but it should be noted that mere possession of such a tripeptide is not necessarily sufficient for the peptide to possess suitable specific binding activity: the inventors have found some examples of peptides which comprise one of the aforementioned tripeptide sequences but which do not exhibit suitable specific binding activity. With the benefit of the present disclosure, those skilled in the art will readily be able to screen candidate peptides and select those having the most desirable binding characteristics.

It will be apparent from the present disclosure that, if using D-isomers of amino acids, the reverse sequences may be employed (i.e. Xaa-Glu-Phe; Asp-Xaa-Phe; and Asp-Glu-Xaa).

U.S. Pat. No. 5,635,182 discloses a 20mer peptide derived from bovine phospholipase C-II, having the sequence QPFEDFRISQEHLADHFDGR.

The present inventors have found that several peptides comprising the tripeptide FED are useful as peptide mimotopes in accordance with the invention. The inventors have not performed any experiments to investigate whether the 20mer disclosed in U.S. Pat. No. 5,635,182 might also be useful (i.e. be capable of binding specifically to an antibody specific to estradiol) but, in the event that the prior art peptide does exhibit such specific binding activity, the inventors hereby provisionally disclaim the peptide consisting of the amino acid sequence QPFEDFRISQEHLADHFDGR.

Purification of the peptide mimotopes can be accomplished by conventional means, such as those described in Tendler et al., The role of the arginine residue in the stabilization of mucin core type 1 β turns. Protein and Peptide Letters, 1, 39-43 (1994). Preferably, the peptide mimotopes will be purified to 95%, optimally to 99%. The mimotope is typically provided as a simple peptide, but may optionally be covalently peptide bonded or linked in some other way to other moieties, such as a label or a solid support.

In one embodiment of the invention, the peptide mimotopes are utilized in an immunoassay test device. Such a device can take different forms, and it can be varied depending on the precise nature of the assay being performed.

Because the peptide mimotopes “mimic” a substance (i.e. Estradiol) which will often be the subject of testing, they are essentially antigenic by nature and function. Thus, it is most preferable that they be utilized in competitive or displacement-type assays (hereinafter collectively referred to as competitive assays). Nothing, however, would preclude their usage in conventional sandwich-type assays as well and specific formats can be readily designed.

Specifically, it is contemplated that in a competitive assay incorporating the peptide mimotopes of the invention, the mimotopes would be coated onto a solid support, typically nitrocellulose or other hydrophobic porous material. They may also be coated on synthetic plastics materials, microtitre assay plates, latex beads, filters comprising cellulosic or synthetic polymeric materials, glass or plastic slides, dipsticks, capillary fill devices and the like.

Coating of the peptide mimotopes to these surfaces can be accomplished by methods known in the art and described in, for example, EP-B-0291194. A particular advantage of the present invention is that unlike the compounds which they mimic, the mimotopes of invention are peptides, and thus can be coated directly onto certain assay surfaces such as nitrocellulose. Estradiol, by contrast, is non-compatible with such cellulosic materials and thus often needs to be bound to the surface by forming a complex with another molecule. Proteins are typically used for such complexing, with BSA often being the most preferred.

In a preferred competitive assay the peptide mimotopes, once coated on the surface of a support, are specifically bound to antibodies or fragments or constructs thereof. The antibodies can be as described above and should be capable of specific binding to estradiol. It is envisioned that a liquid sample containing estradiol migrating over the region containing the antibodies bound to the mimotopes would displace a certain amount of antibodies from the surface of the support. The amount of antibodies displaced would be dependent on several factors including the concentration of the estradiol in the sample, and the relative binding affinities of the mimotopes and estradiol for the antibodies. The amount of antibody displaced could then be measured as a means to determine the relative concentration of estradiol in the sample.

Alternatively and in another preferred embodiment, it is contemplated that the antibodies could be bound to the surface, with the peptide mimotopes being specifically bound to the antibodies and capable of being displaced by estradiol migrating in a sample in contact with (e.g. through) the support. The displacement would generate a measurable signal of the amount of peptide mimotopes displaced and hence the amount of estradiol in the sample.

Other immunoassay test devices contemplated by the invention include those employing, for example, capillary-fill means in which a liquid sample is drawn into a device by capillary action along a suitably-proportioned capillary inlet. Capillary-fill devices which may be adapted for use in the present invention are disclosed, for example, in Shanks et al., U.S. Pat. No. 5,141,868, Shanks et al., EP-A-0422708, and Birch et al., EP-B-0274215.

Devices such as those described in May et al., U.S. Pat. No. 5,622,871 and May et al., U.S. Pat. No. 5,656,503 are also suitable for practice of the immunoassays of the invention. If used, these devices preferably comprise a hollow elongated casing containing the solid support. The solid support communicates indirectly with the exterior of the casing via a bibulous fluid sample receiving member which may or may not protrude from the casing, the solid support and the sample receiving member being linked so as to allow for the fluid sample to migrate between the two by capillary action.

Spatially distant along the solid support from the sample receiving member are the test and, optionally, control zones. Within the test zone, the peptide mimotopes can be bound to an antibody immobilized on the support. Such immobilisation can be accomplished by any number of known means including chemically coupling using, for example, CNBr, carbonyldiimidazole, or tresyl chloride. Alternatively, various “printing” techniques may be used. These include application of liquid antibodies by micro-syringes, direct printing, ink-jet printing, and the like. Chemical or physical treatment of the support prior to application of the antibody is also specifically contemplated, as such may facilitate immobilisation.

The casing in such devices is typically constructed of opaque or translucent material incorporating at least one aperture through which the analytical result may be observed, either by the naked eye or electronic means.

Such devices can be provided to clinical laboratories or as kits suitable for home use, such kits comprising one or more devices individually wrapped in moisture impervious wrapping and packaged together with appropriate instructions to the user.

The sample receiving member can be made from any bibulous, porous or fibrous material capable of absorbing liquid rapidly. The porosity of the material can be unidirectional (i.e. with pores or fibres running wholly or predominantly parallel to an axis of the member) or multidirectional (omnidirectional, so that the member has an amorphous sponge-like structure). Porous plastics material, such as polypropylene, polyethylene (preferably of very high molecular weight), polyvinylidene fluoride, ethylene vinylacetate, acrylonitrile and polytetrafluoro-ethylene can be used. It can be advantageous to pre-treat the member with a surface-active agent during manufacture, as this can reduce any inherent hydrophobicity in the member and therefore enhance its ability to take up and deliver a moist sample rapidly and efficiently. Porous sample receiving members can also be made from paper or other cellulosic materials, such as nitrocellulose. Preferably the material comprising the sample receiving member should be chosen such that the porous member can be saturated with liquid sample within a matter of seconds. The liquid must be capable of permeating freely from the porous sample receiving member into the solid support.

The solid support in such devices is preferably a dry porous carrier. It may be made of separate strips or sheets and, like the sample receiving member, can be constructed from any material capable of allowing the liquid sample to migrate through a portion of its length by, preferably, capillary action. The support should allow for the immobilisation of the antibody and/or peptide mimotope on its surface, and should not interfere with the binding reactions which are necessary for the proper functioning of the assay.

The solid support may have associated with it an absorbent “sink” which will facilitate capillary action of fluid up the length of the support, and will provide a means by which to avoid flooding of the test device by application of excess sample. Specific materials for and applications of sinks are conventional in the art and may be readily applied to the devices of the present invention.

In the immunoassay test devices of the invention, in order to provide a measurable signal of the amount of analyte in the sample it is preferred that either the peptide mimotope, or the antibody to which it is bound, be labelled. In the preferred embodiment of the invention, the label is any entity the presence of which can be readily detected. Preferably the label is a direct label, such as the those described in detail in May et al., U.S. Pat. No. 5,656,503. Direct labels are entities which, in their natural state, are readily visible either to the naked eye, or with the aid of an optical filter and/or applied stimulation, e.g. UV light to promote fluorescence. Examples include radioactive, chemiluminescent, electroactive (such as redox labels), and fluorescent compounds. Direct particulate labels, such as dye sols, metallic sols (e.g. gold) and coloured latex particles, are also very suitable and are, along with fluorescent compounds, preferred. Of these options, coloured latex particles and fluorescent compounds are most preferred. Concentration of the label into a small zone or volume should give rise to a readily detectable signal, e.g. a strongly coloured area.

Indirect labels, such as enzymes, e.g. alkaline phosphatase and horseradish peroxidase, can also be used, but these usually require the addition of one or more developing reagents such as substrates before a visible signal can be detected. Hence, they are less preferred.

Such additional reagents can be incorporated in the solid support of the assay device such that they dissolve or disperse when a liquid sample is applied. Alternatively, the developing reagents can be added to the sample before application of the sample to the solid support.

Conjugation of the label to the peptide mimotope or the antibody can be by covalent or non-covalent (including hydrophobic) bonding, or by adsorption. Techniques for such conjugation are commonplace in the art and may be readily adapted for the particular reagents employed. In the preferred embodiment wherein the label is a coloured latex particle, the label is preferably conjugated to the antibody and it is accomplished through adsorption. Where the label is a fluorescent compound, it is preferred that the label be conjugated to or constructed as part of the antibody.

Upon usage of the test device, the label can provide a test and/or control signal which can be detected from the test and control surfaces by known conventional means. This includes evaluation by the naked eye, or more typically when precise measurements are desired, by appropriate instrumentation. Instrumentation is particularly suitable when the control or test signal is measured by the amount of mass of complex at the control or test surface.

The immunoassay test devices of the invention may be applied to virtually any type of biological or non-biological sample, though liquid biological samples derived from urine or serum are preferred. The samples may be purified or diluted prior to assaying. The term “immunoassay test device” as used herein is also intended to encompass components of immunoassay test devices which may be sold or supplied as separate articles and which require the presence of other components in order to form a working test device. In particular, it is contemplated that the immunoassay test device of the invention may be a dipstick, test stick or the like, which are generally provided as disposable items and may be supplied as separate components.

In a second aspect the invention provides an isolated nucleic acid encoding a peptide mimotope in accordance with the first aspect of the invention. The nucleic acid may be prepared by cloning from a library of sequences or from an organism (e.g. a phage or a bacterium), or prepared by in vitro synthesis using standard techniques (e.g. automated solid phase oligonucleotide synthesisers, which are commercially available from many sources) or, less conveniently, by performance of ligation reactions, ligating together component nucleic acid sequences from different sources. Typically the isolated nucleic acid sequence will be a DNA sequence (but could, conceivably, be a sense RNA sequence) and will comprise a minimum of 9 bases. More typically, the nucleic acid (or rather, that portion thereof which encodes the peptide mimotopes) will comprise between 12 and 90 bases, desirably between 15 and 90, and preferably between 15 and 60 bases. The nucleic acid may advantageously comprise other components, such as promoter, enhancer and terminator sequences, one or more origins of replication, and the like. In addition, the isolated nucleic acid may encode a fusion protein, in which the peptide mimotope is fused (at either the 5′ or 3′ terminus) to another polypeptide moiety such as a polypeptide label. In such embodiments as aforesaid, whilst that portion of the nucleic acid which encodes the mimotope will generally comprise a number of bases within the ranges identified above, the nucleic acid as a whole may be considerably larger. It will be understood that the nucleic acid molecule may, in some embodiments, encode a peptide which consists solely of the peptide mimotope without any extraneous amino acid residues (e.g. the mimotope will be in isolation from the sequences adjacent thereto in any naturally-occurring molecule from which the mimotope may be derived).

The isolated nucleic acid molecule may conveniently take the form of a plasmid or other replicable moiety.

In a third aspect, the invention provides for the use of a peptide mimotope in accordance with the first aspect of the invention defined above, to assay for the presence and/or amount of estradiol in a sample to be tested.

The practice of the invention is described in detail below with reference to specific illustrative examples, but the invention is not to be construed as being limited thereto.

### EXAMPLES

### Identification of Peptide Mimotopes for Estradiol

Means by which to identify examples of peptide mimotopes of the estradiol metabolite, estrone-3-glucuronide, are described below.

Monoclonal antibodies MAb 4155 were expressed from the 4155 monoclonal cell line. The 4155 monoclonal cell line was prepared and screened according to the methods described by Gani et al., (J Steroid Biochem. Molec. Biol. 48, 277-282 (1994)). The Gani et al. publication relates to development of anti-progesterone antibodies, but similar techniques were employed in producing antibodies reacting with estrone and analogues thereof.

Comparative amino acid sequences utilized in the following examples are as follows:

| | |
|---|---|
| Glu-Asp | (SEQ ID NO:5) |
| Ala-Ala-Glu-Arg-Gly-Leu-Phe-Glu | (SEQ ID NO:71) |
| Ala-Ala-Glu-Arg-Gly-Leu-Phe | (SEQ ID NO:72) |
| Ala-Ala-Glu-Arg-Gly-Leu | (SEQ ID NO:73) |
| Ala-Ala-Glu-Arg-Gly | (SEQ ID NO:74) |
| Ala-Ala-Glu-Arg | (SEQ ID NO:75) |
| Ala-Ala-Glu | (SEQ ID NO:76) |
| Ala-Ala | (SEQ ID NO:77) |
| Ala-Ala-Glu-Arg-Gly-Leu-Ala-Glu-Asp | (SEQ ID NO:78) |
| Ala-Ala-Glu-Arg-Gly-Leu-Phe-Ala-Asp | (SEQ ID NO:79) |
| Ala-Ala-Glu-Arg-Gly-Leu-Phe-Glu-Ala | (SEQ ID NO:80) |

### Example 1

### Identification of Peptide Mimotope Sequences by Phage Display

pVIII9aa-cys Nonapeptide Phage Library

The VIII9aa-cys library phage library described by Felici F et al., Mimicking of discontinuous epitopes by phage-displayed peptides, II. Selection of clones recognised by a protective monoclonal antibody against the Bordetella pertussis toxin from phage peptide libraries. Gene 128, 21-27 (1993) and Luzzago et al. Mimicking of discontinuous epitopes by phage-displayed peptides, I. Epitope mapping of human H ferritin using a phage library of constrained peptides. Gene 128, 51-57 (1993) was used. The library consisted of random nonapeptides fused to the major coat protein pVIII so that several hundred peptides were displayed on each phage particle.

### Screening of Phage Library

Affinity selection of phage was performed by a combination of the methods of Folgori A. et al. A general strategy to identify mimotopes of pathological antigens using only random peptide libraries and human sera. EMBO J 13, 2236-2243 (1994) and Parmley S. F. et al. Antibody-selectable fd phage vectors:affinity purification of target genes. Gene 73, 305-318 (1988).

Polystyrene tubes used for panning (Immunotubes™ from Nunc) were coated either with affinity-purified anti-estrone-3-glucuronide antibodies (20 μg) in 2 mls of coating buffer (0.1 M NaHCO₃ , pH 9.0) or with coating buffer only overnight at 4° C. After three washes with tris buffered saline (TBS; 50 mM tris-HCl, 140 mM NaCl, pH 7.4) both tubes were incubated with 4 mls of blocking buffer (TBS containing 10 mg/ml ovalbumin) for 4 h at room temperature. The VIII9aa-cys library was shown to have a titre of 1×10¹³ transducing units/ml (TU/ml) by infection of logarithmic XL1-Blue bacteria (Stratagene, Amsterdam, Holland). Aliquots (1 μl; 1×10¹¹ TU) from the donated phage suspension were added to the antibody-coated and un-coated polystyrene tubes each containing 1 ml of TBS and 1 mg/ml ovalbumin and incubated overnight at 4° C. Unbound phage were removed by 15 washes (each of 4 ml) with TBS containing 0.5% (v/v) Tween 20™ (TTBS) followed by 5 washes with TBS at room temperature. Bound phage were eluted by incubation of washed panning tubes with 1 ml of elution buffer (0.1 M HCl, pH 2.2, adjusted with glycine, containing 1 mg/ml ovalbumin) for 12 min at room temperature. The eluted phage were transferred to 1 ml polypropylene tubes and neutralised with 60 μl of 2 M tris (pH not adjusted). Aliquots (200 μl) of 1 M tris-HCl, pH 7.4 were also added to the panning tubes for neutralisation. The eluted neutralised phage particles (1 ml) were used for infection of 9 ml of logarithmic XL1-Blue bacteria (in 2TY containing 1% (w/v) glucose). Logarithmic XL1-Blue bacteria (4 ml) were also added directly to the neutralised panning tubes. Infection was carried out for 30 min at 37° C. with no shaking. The infected bacteria were then pooled (total volume 13 ml) ampicillin was added (to 100 μg/ml) and the cultures incubated overnight with shaking at 37° C. A small aliquot (10 μl) of infected bacterial cells was removed prior to overnight incubation for titration (diluted 10⁻² to 10⁻⁶ in 2TY/Amp/Glucose) on 2TY agar containing 1% (w/v) glucose and ampicillin (100 μg/ml). An aliquot (150 μl) of the overnight XL1-Blue culture infected with phage eluted from the panning tube coated with MAb 4155 antibodies was then added to 15 ml of 2TY containing 1% (w/v) glucose and 100 μg/ml ampicillin and grown to logarithmic phase. The cells were then superinfected with M13K07 helper phage (Gibco BRL Life Technologies, Paisley, Scotland) (1×10¹¹ phage/ml) and incubated for 30 min without shaking at 37° C. This was followed by centrifugation for 20 min at 1800 rpm and resuspension of the cell pellet in 200 ml of 2TY containing 100 μg/ml ampicillin and 25 μgm/ml kanamycin. The bacterial culture was then incubated overnight at 37° C. with shaking. Bacterial cells were then pelleted by centrifugation (5000 rpm; 15 min) and the phage particles in the supernatant precipitated by addition of 40 mls of PEG/NaCl (2.5M NaCl containing 20% (w/v) polyethylene glycol 8000) and incubation on ice for 1 hour. The phage suspension was then spun at 10,000 rpm for 20 min at 4° C. and the resulting pellet resuspended in 20 ml of TBS. A further PEG precipitation was carried out by addition of 4 ml of PEG/NaCl and incubation on ice for a further 20 min. The final phage pellet was dissolved in 2 ml of TBS which resulted in phage titres of the order of 1×10¹³ TU/ml. These phage particles were added directly to fresh panning tubes and the entire panning procedure repeated a further two times. The entire screening protocol (three rounds of panning) was repeated after the first screen.

The output from the third round of panning was plated out on 2TY agar, ampicillin (100 μg/ml) and 1% (w/v) glucose and incubated overnight at 37° C. Random individual bacterial colonies (˜200) were picked and added to the wells of 96-well microtitre plates (Sterilin™) each containing 200 μl of 2TY, 1% (w/v) glucose and ampicillin (100 μg/ml). The microtitre plates were incubated overnight with shaking at 37° C. The following day aliquots from each well (20 μl) were added to the wells of fresh microtitre plates each containing 200 μl of 2TY, 1% glucose, 100 μg/ml ampicillin and incubated with shaking for 1 h at 37° C. At the next stage, 25 μl of 2TY containing ampicillin (100 μg/ml), 1% (w/v) glucose and 10⁹ M13KO7 helper phage were added to each well. The plates were incubated for 30 min at 37° C. without shaking followed by a further incubation for 1 h at 37° C. with shaking. The plates were then spun at 1800 rpm for 20 min at room temperature, the supernatant aspirated off and the cell pellet resuspended in 200 μl of 2TY containing ampicillin (100 μg/ml) and kanamycin (20 μg/ml). Incubation with shaking at 37° C. was then carried out overnight. Centrifugation of overnight cultures in the wells of microtitre plates was carried out (1500 rpm; 20 mins) and phage-containing supernatants (100 μl) were added to sheep anti-M13 bacteriophage (C.P. Laboratories, Bishops Stortford, UK) coated microtitre plates (Greiner™, high bind). Purified sheep anti-M13 antibody-coated plates were prepared by overnight incubation (100 μl/well; 10 μg/ml) at 4° C. in binding buffer (0.1 M NaHCO₃, pH 9.0). Blocking was carried out with PBST containing 10 mg/ml ovalbumin (200 μl/well) for 1 h at room temperature. After removal of unbound phage from sheep anti-M13-coated plates by five washes with PBST affinity-purified anti-estrone-3-glucuronide antibodies were added (20 μg) in 2 mls of PBST containing 10 mg/ml ovalbumin; 100 μl per well). Incubation was carried out for 2 h at room temperature. Alkaline phosphatase conjugated rabbit anti-mouse immunoglobulin (100 μl/well) was then added at a dilution of 1/1000 (in PBST, 10 mg/ml ovalbumin) and incubated for a further 2 h at room temperature. The assay was developed with 100 μl/well of p-nitrophenyl phosphate (1 mg/ml) in 1M diethanolamine, 1 mM MgCl₂, pH 9.6 and the plates read at 410 nm.

### DNA Sequencing

Double-stranded phagemid DNA was purified from bacterial cultures (50 ml) infected with positive phage clones using the Qiagen™ plasmid purification kit according to the manufacturer's instructions. Sequencing was carried out on an Applied Biosystems automated sequencer (Model 373A, version 1.2.0) using the oligonucleotide primer SEQ ID NO 1:

| | |
|---|---|
| 5′- TTT CCC AGT CAC GAC GTT G -3′. | (SEQ ID NO:1) |

From the Phage ELISA and DNA sequencing results the following three peptide mimotope sequences were identified:

| | |
|---|---|
| Ala-Ala-Glu-Arg-Gly-Leu-Phe-Glu-Asp. | (SEQ ID NO:2) |
| Thr-Ala-Trp-Thr-Tyr-Val-Leu-Gly-Phe. | (SEQ ID NO:3) |
| Thr-Ser-Trp-Ala-Tyr-Val-Leu-Gly-Pro. | (SEQ ID NO:4) |

### Identification of Mimotope Core Binding Regions by Replacement Net Analysis

### Solid Phase Peptide Synthesis on Pins

Peptides were synthesised in duplicate or triplicate from the C-terminus by solid phase peptide synthesis on the heads of polyethylene pins (Geysen et al., Strategies for epitope analysis using peptide synthesis. J. Immunol. Methods 102, 259-274 (1987)) using a Multipin Peptide Synthesis Kit (Chiron Mimotopes, Victoria, Australia). Pins were arranged in a plastic holder in the format of a 96-well microtitre plate.

### ELISA Testing of Peptides on Pins

All incubation steps were performed at room temperature (18-25° C.) by lowering the pins reagents dispensed into 96-well microtitre plates (Becton Dickinson, CA, USA). Washing was accomplished by placing the block of pins in a bath of phosphate buffered saline (PBS) containing Tween 20™ (0.01% v/v) with agitation for four cycles of 5 min. Non-specific binding sites on the surface of the pins were blocked by incubating in PBS containing casein (1% w/v, 175 μl/well) for 1 h. MAb4155 was diluted in blocking buffer and the pins were incubated in the antibody solution (150 μl/well) for 18 h at 4° C. After washing, the pins were incubated in horseradish peroxidase (HRP)-conjugated rabbit anti-mouse immunoglobulin (Dako, High Wycombe, UK, 1/1000 in blocking buffer for 1 h at 150 μl/well). The pins were washed once more and then incubated in ABTS [2,2′-azino-bis (3-ethylbenzothiazoline-6-sulphonic acid)] working substrate for 15 min (150 μl/well). ABTS was prepared as a 0.033% (w/v) solution in 0.1M citrate phosphate buffer (pH 4.5) with 33% hydrogen peroxide (1 μl/ml). Colour development was terminated by removal of the pins from the wells and was measured spectrophotometrically at 405 nm using a Milenia Kinetic Analyser™ (DPC, Llanberis, Wales).

### Identification of the Core Binding Regions

In order to identify core binding regions, a set of peptides was synthesised on the heads of pins based on SEQ ID NO:2. These included peptides sequentially reduced in length by one amino acid, first from the N-terminus and then, in another series, from the C-terminus. In addition, a set of peptides was synthesised in which each residue of the lead sequence was replaced by Ala (or Gly if Ala already existed at that position) in order to assess the contribution of each residue to the binding event. MAb4155 was tested for reactivity with these peptides, the results being shown below in Table 1.

TABLE 1

| | | | |
|---|---|---|---|
| | SEQ ID | | Relative |
| Mimotope | NO: | Type | Binding* |

| | | | |
|---|---|---|---|
| Ala-Ala-Glu-Arg-Gly-Leu- | 2 | invention | 2.0 |
| Phe-Glu-Asp | | | |
| Ala-Glu-Arg-Gly-Leu-Phe- | 11 | invention | 1.5 |
| Glu-Asp | | | |
| Glu-Arg-Gly-Leu-Phe-Glu- | 10 | invention | 1.8 |
| Asp | | | |
| Arg-Gly-Leu-Phe-Glu-Asp | 9 | invention | 1.6 |
| Gly-Leu-Phe-Glu-Asp | 8 | invention | 2.2 |
| Leu-Phe-Glu-Asp | 7 | invention | 1.9 |
| Phe-Glu-Asp | 6 | invention | 2.0 |
| Gln-Asp | 5 | comparison | 1.0 |
| Ala-Ala-Glu-Arg-Gly-Leu- | 71 | comparison | 1.0 |
| Phe-Glu | | | |
| Ala-Ala-Glu-Arg-Gly-Leu- | 72 | comparison | 1.0 |
| Phe | | | |
| Ala-Ala-Glu-Arg-Gly-Leu | 73 | comparison | 1.0 |
| Ala-Ala-Glu-Arg-Gly | 74 | comparison | 1.0 |
| Ala-Ala-Glu-Arg | 75 | comparison | 0.8 |
| Ala-Ala-Glu | 76 | comparison | 0.9 |
| Ala-Ala | 77 | comparison | 1.0 |
| Gly-Ala-Glu-Arg-Gly-Leu- | 12 | invention | 1.9 |
| Phe-Glu-Asp | | | |
| Ala-Gly-Glu-Arg-Gly-Leu- | 13 | invention | 2.0 |
| Phe-Glu-Asp | | | |
| Ala-Ala-Ala-Arg-Gly-Leu- | 14 | invention | 1.5 |
| Phe-Glu-Asp | | | |
| Ala-Ala-Glu-Ala-Gly-Leu- | 15 | invention | 2.0 |
| Phe-Glu-Asp | | | |
| Ala-Ala-Glu-Arg-Ala-Leu- | 16 | invention | 1.5 |
| Phe-Glu-Asp | | | |
| Ala-Ala-Glu-Arg-Gly-Ala- | 17 | invention | 1.6 |
| Phe-Glu-Asp | | | |
| Ala-Ala-Glu-Arg-Gly-Leu- | 78 | comparison | 0.8 |
| Ala-Glu-Asp | | | |
| Ala-Ala-Glu-Arg-Gly-Leu- | 79 | comparison | 1.8 |
| Phe-Ala-Asp | | | |
| Ala-Ala-Glu-Arg-Gly-Leu- | 80 | comparison | 11.1 |
| Phe-Glu-Ala | | | |

* Relative Binding of MAb4155 to peptides as measured by ELISA as described above

As is demonstrated from the data, of the peptides tested, only those amino acid sequences comprising the core binding region as indicated by SEQ ID NO:6 provided adequate binding. Amino acid sequences represented by SEQ ID NO:7-17 contain the core binding region of SEQ ID NO:6 and provided adequate binding to serve as an estradiol mimotope.

| | |
|---|---|
| Phe-Glu-Asp | (SEQ ID NO:6) |
| Leu-Phe-Glu-Asp | (SEQ ID NO:7) |
| Gly-Leu-Phe-Glu-Asp | (SEQ ID NO:8) |
| Arg-Gly-Leu-Phe-Glu-Asp | (SEQ ID NO:9) |
| Glu-Arg-Gly-Leu-Phe-Glu-Asp | (SEQ ID NO:10) |
| Ala-Glu-Arg-Gly-Leu-Phe-Glu-Asp | (SEQ ID NO:11) |
| Gly-Ala-Glu-Arg-Gly-Leu-Phe-Glu-Asp | (SEQ ID NO:12) |
| Ala-Gly-Glu-Arg-Gly-Leu-Phe-Glu-Asp | (SEQ ID NO:13) |
| Ala-Ala-Ala-Arg-Gly-Leu-Phe-Glu-Asp | (SEQ ID NO:14) |
| Ala-Ala-Glu-Ala-Gly-Leu-Phe-Glu-Asp | (SEQ ID NO:15) |
| Ala-Ala-Glu-Arg-Ala-Leu-Phe-Glu-Asp | (SEQ ID NO:16) |
| Ala-Ala-Glu-Arg-Gly-Ala-Phe-Glu-Asp | (SEQ ID NO:17) |

Additional core binding sequences were identified utilizing the amino acid sequence SEQ ID NO:8 and investigating the effect of systematic replacement of each residue by the other 19 naturally occurring amino acids using known techniques as exemplified in Verhoeyen et al., Construction of a reshaped HMFG1 antibody and comparison of its fine specificity with that of the parent mouse antibody. Immunology, 78, 364-370 (1993).

The sequences which had superior binding reactivity and specificity compared to SEQ ID NO:8 are identified as follows. The binding of MAb4155 to these sequences as determined by ELISA is shown below in Table 2, setting SEQ ID NO:8 to a Relative Binding of 100.

| | |
|---|---|
| Gly-Phe-Phe-Glu-Asp | (SEQ ID NO:18) |
| Gly-Trp-Phe-Glu-Asp | (SEQ ID NO:19) |
| Gly-Tyr-Phe-Glu-Asp | (SEQ ID NO:20) |
| Gly-Leu-Trp-Glu-Asp | (SEQ ID NO:21) |
| Gly-Leu-Phe-Cys-Asp | (SEQ ID NO:22) |
| Gly-Leu-Phe-Asp-Asp | (SEQ ID NO:23) |
| Gly-Leu-Phe-Phe-Asp | (SEQ ID NO:24) |
| Gly-Leu-Phe-Ile-Asp | (SEQ ID NO:25) |
| Gly-Leu-Phe-Leu-Asp | (SEQ ID NO:26) |
| Gly-Leu-Phe-Trp-Asp | (SEQ ID NO:27) |
| Gly-Leu-Phe-Tyr-Asp | (SEQ ID NO:28) |
| Gly-Leu-Phe-Glu-Cys | (SEQ ID NO:29) |
| Gly-Leu-Phe-Glu-Phe | (SEQ ID NO:30) |
| GIy-Leu-Phe-Glu-Ile | (SEQ ID NO:31) |
| Gly-Leu-Phe-Glu-Leu | (SEQ ID NO:32) |
| Gly-Leu-Phe-Glu-Val | (SEQ ID NO:33) |
| Gly-Leu-Phe-Glu-Trp | (SEQ ID NO:34) |
| Gly-Leu-Phe-Glu-Tyr | (SEQ ID NO:35) |

TABLE 2

| | | |
|---|---|---|
| Mimotope | SEQ ID NO: | Relative |

| | | |
|---|---|---|
| Gly-Leu-Phe-Glu-Asp | 8 | 100 |
| Gly-Phe-Phe-Glu-Asp | 118 | 200 |
| Gly-Trp-Phe-Glu-Asp | 119 | 343 |
| Gly-Tyr-Phe-Glu-Asp | 20 | 220 |
| Gly-Leu-Trp-Glu-Asp | 21 | 207 |
| Gly-Leu-Phe-Cys-Asp | 22 | 335 |
| Gly-Leu-Phe-Asp-Asp | 23 | 121 |
| Gly-Leu-Phe-Phe-Asp | 24 | 184 |
| Gly-Leu-Phe-Ile-Asp | 25 | 169 |
| Gly-Leu-Phe-Leu-Asp | 26 | 138 |
| Gly-Leu-Phe-Trp-Asp | 27 | 578 |
| Gly-Leu-Phe-Tyr-Asp | 28 | 252 |
| Gly-Leu-Phe-Glu-Cys | 29 | 296 |
| Gly-Leu-Phe-Glu-Phe | 30 | 204 |
| Gly-Leu-Phe-Glu-Ile | 31 | 174 |
| Gly-Leu-Phe-Glu-Leu | 32 | 168 |
| Gly-Leu-Phe-Glu-Val | 33 | 177 |
| Gly-Leu-Phe-Glu-Trp | 34 | 594 |
| Gly-Leu-Phe-Glu-Tyr | 35 | 386 |

* Relative Binding of MAb4155 to peptides as measured by ELISA as described above

### D-isomers of the Reverse Sequence of the Core Binding Regions.

The foregoing SEQ ID NO.'s 1-35 are L-isomers. It was also demonstrated that the D-isomers of the reverse sequences of those core binding regions identified above similarly function as effective mimotopes. For example, an amino acid sequence as described by SEQ ID NO:37 was prepared by the peptide synthesis methods described above. Binding affinity relative to the parent sequence (SEQ ID NO:28) was measured by the described testing methods. The results show the reverse sequence to have an equivalent relative binding affinity compared to the parent sequence.

The following therefore identify core binding sequences of peptides capable of functioning as mimotopes for estradiol. Each sequence contains D-isomers of the amino acids in the reverse sequence of one of those described above.

### Note: SEQ ID NO:s 36-56 are D-isomers

| | |
|---|---|
| Asp-Glu-Phe | (SEQ ID NO:36) |
| Asp-Tyr-Phe-Leu-Gly | (SEQ ID NO:37) |
| Asp-Glu-Phe-Phe-Gly | (SEQ ID NO:38) |
| Asp-Glu-Phe-Trp-Gly | (SEQ ID NO:39) |
| Asp-Glu-Phe-Tyr-Gly | (SEQ ID NO:40) |
| Asp-Glu-Trp-Leu-Gly | (SEQ ID NO:41) |
| Asp-Cys-Phe-Leu-Gly | (SEQ ID NO:42) |
| Asp-Asp-Phe-Leu-Gly | (SEQ ID NO:43) |
| Asp-Phe-Phe-Leu-Gly | (SEQ ID NO:44) |
| Asp-Ile-Phe-Leu-Gly | (SEQ ID NO:45) |
| Asp-Leu-Phe-Leu-Gly | (SEQ ID NO:46) |
| Asp-Trp-Phe-Leu-Gly | (SEQ ID NO:47) |
| Cys-Glu-Phe-Leu-Gly | (SEQ ID NO:48) |
| Phe-Glu-Phe-Leu-Gly | (SEQ ID NO:49) |
| Ile-Glu-Phe-Leu-Gly | (SEQ ID NO:50) |
| Leu-Glu-Phe-Leu-Gly | (SEQ ID NO:51) |
| Val-Glu-Phe-Leu-Gly | (SEQ ID NO:52) |
| Trp-Glu-Phe-Leu-Gly | (SEQ ID NO:53) |
| Tyr-Glu-Phe-Leu-Gly | (SEQ ID NO:54) |
| Phe-Gly-Leu-Val-Tyr-Thr-Trp-Ala-Thr | (SEQ ID NO:55) |
| Pro-Gly-Leu-Val-Tyr-Ala-Trp-Ser-Thr | (SEQ ID NO:56) |

### Example 2

Identification of Peptide Mimotope Sequences from Pepscan Libraries

The pins from the Multipin Peptide Synthesis Kit as described in Example 1 were used to construct libraries of peptide sequences encompassing all possible trimer combinations of the 20 naturally occurring amino acids, supplemented by a further random set of dodecapeptides (Slootstra J W et al., Screening of a small set of random peptides: a new strategy to identify synthetic peptides that mimic epitopes J Molec. Recog. 10, 217-224 (1997)). The binding of MAb4155 was tested on the library for binding affinity in a manner as described in Example 1. This identified the following amino acid sequences (L-isomers) as core binding regions for estradiol mimotopes.

| | |
|---|---|
| Asp-Phe-Tyr | (SEQ ID NO:57) |
| Phe-Tyr-Glu | (SEQ ID NO:58) |
| Tyr-Glu-Glu | (SEQ ID NO:59) |
| Tyr-Gln-Glu | (SEQ ID NO:60) |
| Asn-Glu-Glu-Asp-Phe-Tyr-Gln-Ile-Gln- | (SEQ ID NO:61) |
| Leu-Tyr-Glu | |
| Arg-Gln-Ile-Asp-Phe-Tyr-Gln-Glu-Ile- | (SEQ ID NO:62) |
| Gln-Phe-Lys | |
| Asp-Asp-Phe-Tyr-Gly-Gln-Pro-Arg-Glu- | (SEQ ID NO:63) |
| Gln-Val-Arg | |

Similarly to Example 1 the following reverse sequences of D-amino acids are identified as capable of functioning as the core binding region for peptide mimotopes for estradiol.

### Note: SEQ ID NO:s 64-70 are D-isomers

| | |
|---|---|
| Tyr-Phe-Asp | (SEQ ID NO:64) |
| Glu-Tyr-Phe | (SEQ ID NO:65) |
| Glu-Glu-Tyr | (SEQ ID NO:66) |
| Glu-Gln-Tyr | (SEQ ID NO:67) |
| Glu-Tyr-Leu-Gln-Ile-Gln-Tyr-Phe-Asp- | (SEQ ID NO:68) |
| Glu-Glu-Asn | |
| Lys-Phe-Gln-Ile-Glu-Gln-Tyr-Phe-Asp- | (SEQ ID NO:69) |
| Ile-Gln-Arg | |
| Arg-Val-Gln-Glu-Arg-Pro-Gln-Gly-Tyr- | (SEQ ID NO:70) |
| Phe-Asp-Asp | |

### Example 3

### Comparison of Competitive Assays for Estrone-3-Glucuronide (E3G) Using E3G or a Peptide Mimotope of E3G on a Solid Phase

### Peptide Ligand Synthesis

Synthetic peptide ligands were prepared on an Applied Biosystems 431A Peptide Synthesiser Biopolymer Synthesis and Analysis Unit, TM QMC, (Nottingham, UK). Purity was assessed by mass spectroscopy and HPLC and was in excess of 95%

### Preparation of Ovalbumin-E3G Conjugate

An estrone-3-glucuronide (E3G) ovalbumin conjugate was prepared by resuspending 2.6 mg of E3G in 2 ml of freshly prepared solution of EDC (1-ethyl (dimethylaminopropyl) carbodiimide, 0.1 M) and NHS (N-hydroxysuccinamide, 0.02M) and incubating for 15 minutes at room temperature. To the E3G solution, 2 ml of ovalbumin (10 mg/ml) was added and this was incubated for 2.5 hrs at room temperature with constant mixing. The conjugate was then dialysed for 16 hrs against 1 L of phosphate buffered saline containing 0.1% sodium azide.

### Preparation of BSA-mimotope Conjugate

Bovine serum albumin (BSA, 10 mg, Sigma) was dissolved in 3 ml of conjugation buffer (sodium hydrogen carbonate buffer, 0.1M, pH 8.4) in a clean glass vial, mixing by suction and expulsion from a pipette tip. The mixture was left on a roller for one hour. The peptide mimotope as represented by SEQ ID NO:36 was dissolved in conjugation buffer. Peptide solution (1.0 ml@10 mg/ml) and 10 μl glutaraldehyde (high commercial grade, Sigma) were added to the BSA solution. The sealed vial was then agitated on a roller for four hours at room temperature. The conjugate solution was then dialysed against sodium chloride (0.9% w/v) for 48 h at 4° C.

### Assays:

Peptide-mimotope-BSA conjugate (10 μg/ml) and E3G-ovalbumin conjugate (3 μg/ml) were dried separately into the wells of a microtitre plate (Becton Dickinson, CA, USA) from 50 μl of solution in Phosphate Buffered Saline (PBS) overnight at room temperature. Wells were washed (4× PBS+0.01% Tween20™), blocked for 1 hour with 0.1% casein in 100 μl of PBS and washed 4× before use. 25 μl aliquots of E3G in PBS (0-3 uM) were added and incubated for 15 mins before adding in 25 μl of the MAb4155 anti-E3G antibody at 0.6 μg/ml in PBS. The wells were incubated, with agitation, for 1 hour at room temperature. After washing 4×, 50 μl of rabbit anti-mouse IgG-HRP conjugate (Dako, High Wycombe, UK) at 1:1000 dilution in PBS was added to each well and incubated for 1 hour at room temperature. After washing 4×, the wells were incubated in ABTS [2,2′-azino-bis (3-ethylbenzothiazoline-6-sμlphonic acid)] working substrate for 15 min (150 μl/well). ABTS was prepared as a 0.033% (w/v) solution in 0.1M citrate phosphate buffer (pH 4.5) with 33% hydrogen peroxide (1 μ/ml). Colour development was terminated by addition of 0.5M sulphuric acid (10 μ/well) and was measured spectrophotometrically at 405 nm using a Milenia Kinetic Analyser™ (DPC, Llanberis, Wales).

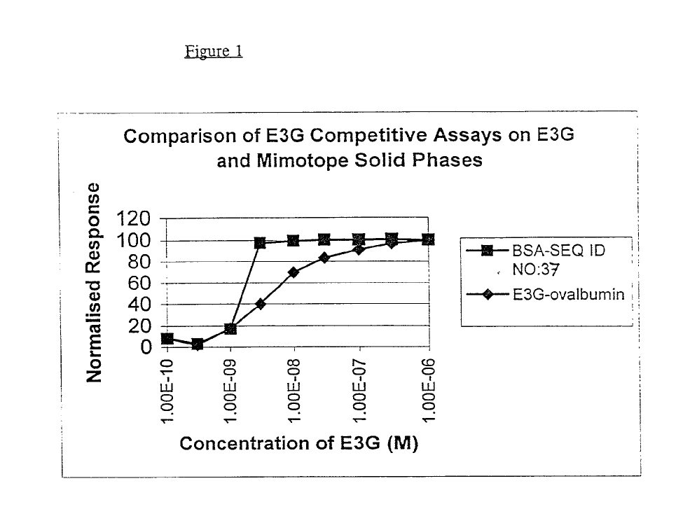

FIG. 1 shows the resulting assay curves. Both are typical for competitive immunoassays having midpoints in the micromolar to nanomolar range. Furthermore, the assay sensitivity using the mimotope-containing BSA-SEQ ID NO:37 is significantly greater than that obtained when using the epitope-containing E3G-ovalbumin.

The invention has been described in detail with particular reference to preferred embodiments thereof, but it will be understood that variations and modifications can be effected with the spirit and scope of the invention.

1. A purified peptide mimotope which is capable of binding specifically to an antibody specific to estradiol.

2. A purified peptide mimotope according to claim 1, wherein the mimotope has a core binding region of no greater than 12 amino acid residues.

3. A purified peptide mimotope according to claim 1, having a core binding region represented by an amino acid sequence selected from the group consisting of SEQ ID NO's: 3, 4, 6 and 18-70.

4. A purified peptide mimotope according to claim 1, which binds specifically to an antibody specific to estrone-3-glucuronide.

5. A purified peptide mimotope according to claim 1, comprising an amino acid sequence selected from the group consisting of SEQ ID NO's: 7-17.

6. A purified peptide according to claim 1 comprising, in its core binding region, a tripeptide in accordance with at least one of the following: Xaa-Glu-Asp; Phe-Xaa-Asp; and Phe-Glu-Xaa.

7. A solid support having immobilised (releasably or non-releasably) thereon a purified peptide in accordance with claim 1.

8. A solid support according to claim 7, in the form of an immunoassay test stick or dipstick.

9. An immunoassay test device for the detection in a sample of estradiol, the immunoassay comprising a peptide mimotope according to claim 1, and an antibody capable of binding specifically to the peptide mimotope to generate a detectable signal.

10. An immunoassay test device according to claim 9, wherein the test device is a competitive immunoassay test device.

11. An immunoassay test device according to claim 9, wherein the peptide mimotope comprises an amino acid sequence having a core binding region selected from the group consisting of SEQ ID NO's: 3, 4 and 6 and 18-70.

12. An immunoassay test device according to claim 9, wherein the peptide mimotope comprises an amino acid sequence selected from the group consisting of SEQ ID NO's: 7-17.

13. An immunoassay test device according to claim 9, wherein the peptide mimotope binds specifically to an antibody specific to estrone-3-glucuronide.

14. Use of a peptide mimotope according to claim 1, to assay for the presence and/or amount of estradiol in a sample to be tested.

15. An isolated nucleic acid encoding a peptide mimotope in accordance with claim 1.


SEQUENCE LISTING (80 sequences)

SEQ ID NO: 1 - DNA, 19 bp, artificial sequence
  Synthetic olligonucleotide
tttcccagtcacgacgttg

SEQ ID NO: 2 - protein, 9 aa, artificial sequence
  synthetic peptide
AAERGLFED

SEQ ID NO: 3 - protein, 9 aa, artificial sequence
  synthetic peptide
TAWTYVLGF

SEQ ID NO: 4 - protein, 9 aa, artificial sequence
  synthetic peptide
TSWAYVLGP

SEQ ID NO: 5 - protein, 2 aa, artificial sequence
  synthetic peptide
ED

SEQ ID NO: 6 - protein, 3 aa, artificial sequence
  synthetic peptide
FED

SEQ ID NO: 7 - protein, 4 aa, artificial sequence
  synthetic peptide
LFED

SEQ ID NO: 8 - protein, 5 aa, artificial sequence
  synthetic peptide
GLFED

SEQ ID NO: 9 - protein, 6 aa, artificial sequence
  synthetic peptide
RGLFED

SEQ ID NO: 10 - protein, 7 aa, artificial sequence
  synthetic peptide
ERGLFED

SEQ ID NO: 11 - protein, 8 aa, artificial sequence
  synthetic peptide
AERGLFED

SEQ ID NO: 12 - protein, 9 aa, artificial sequence
  synthetic peptide
GAERGLFED

SEQ ID NO: 13 - protein, 9 aa, artificial sequence
  synthetic peptide
AGERGLFED

SEQ ID NO: 14 - protein, 9 aa, artificial sequence
  synthetic peptide
AAARGLFED

SEQ ID NO: 15 - protein, 9 aa, artificial sequence
  synthetic peptide
AAEAGLFED

SEQ ID NO: 16 - protein, 9 aa, artificial sequence
  synthetic peptide
AAERALFED

SEQ ID NO: 17 - protein, 9 aa, artificial sequence
  synthetic peptide
AAERGAFED

SEQ ID NO: 18 - protein, 5 aa, artificial sequence
  synthetic peptide
GFFED

SEQ ID NO: 19 - protein, 5 aa, artificial sequence
  synthetic peptide
GWFED

SEQ ID NO: 20 - protein, 5 aa, artificial sequence
  synthetic peptide
GYFED

SEQ ID NO: 21 - protein, 5 aa, artificial sequence
  synthetic peptide
GLWED

SEQ ID NO: 22 - protein, 5 aa, artificial sequence
  synthetic peptide
GLFCD

SEQ ID NO: 23 - protein, 5 aa, artificial sequence
  synthetic peptide
GLFDD

SEQ ID NO: 24 - protein, 5 aa, artificial sequence
  synthetic peptide
GLFFD

SEQ ID NO: 25 - protein, 5 aa, artificial sequence
  synthetic peptide
GLFID

SEQ ID NO: 26 - protein, 5 aa, artificial sequence
  synthetic peptide
GLFLD

SEQ ID NO: 27 - protein, 5 aa, artificial sequence
  synthetic peptide
GLFWD

SEQ ID NO: 28 - protein, 5 aa, artificial sequence
  synthetic peptide
GLFYD

SEQ ID NO: 29 - protein, 5 aa, artificial sequence
  synthetic peptide
GLFEC

SEQ ID NO: 30 - protein, 5 aa, artificial sequence
  synthetic peptide
GLFEF

SEQ ID NO: 31 - protein, 5 aa, artificial sequence
  synthetic peptide
GLFEI

SEQ ID NO: 32 - protein, 5 aa, artificial sequence
  synthetic peptide
GLFEL

SEQ ID NO: 33 - protein, 5 aa, artificial sequence
  synthetic peptide
GLFEV

SEQ ID NO: 34 - protein, 5 aa, artificial sequence
  synthetic peptide
GLFEW

SEQ ID NO: 35 - protein, 5 aa, artificial sequence
  synthetic peptide
GLFEY

SEQ ID NO: 36 - protein, 3 aa, artificial sequence
  synthetic peptide
DEF

SEQ ID NO: 37 - protein, 5 aa, artificial sequence
  synthetic peptide
DYFLG

SEQ ID NO: 38 - protein, 5 aa, artificial sequence
  synthetic peptide
DEFFG

SEQ ID NO: 39 - protein, 5 aa, artificial sequence
  synthetic peptide
DEFWG

SEQ ID NO: 40 - protein, 5 aa, artificial sequence
  synthetic peptide
DEFYG

SEQ ID NO: 41 - protein, 5 aa, artificial sequence
  synthetic peptide
DEWLG

SEQ ID NO: 42 - protein, 5 aa, artificial sequence
  synthetic peptide
DCFLG

SEQ ID NO: 43 - protein, 5 aa, artificial sequence
  synthetic peptide
DDFLG

SEQ ID NO: 44 - protein, 5 aa, artificial sequence
  synthetic peptide
DFFLG

SEQ ID NO: 45 - protein, 5 aa, artificial sequence
  synthetic peptide
DIFLG

SEQ ID NO: 46 - protein, 5 aa, artificial sequence
  synthetic peptide
DLFLG

SEQ ID NO: 47 - protein, 5 aa, artificial sequence
  synthetic peptide
DWFLG

SEQ ID NO: 48 - protein, 5 aa, artificial sequence
  synthetic peptide
CEFLG

SEQ ID NO: 49 - protein, 5 aa, artificial sequence
  synthetic peptide
FEFLG

SEQ ID NO: 50 - protein, 5 aa, artificial sequence
  synthetic peptide
IEFLG

SEQ ID NO: 51 - protein, 5 aa, artificial sequence
  synthetic peptide
LEFLG

SEQ ID NO: 52 - protein, 5 aa, artificial sequence
  synthetic peptide
VEFLG

SEQ ID NO: 53 - protein, 5 aa, artificial sequence
  synthetic peptide
WEFLG

SEQ ID NO: 54 - protein, 5 aa, artificial sequence
  synthetic peptide
YEFLG

SEQ ID NO: 55 - protein, 9 aa, artificial sequence
  synthetic peptide
FGLVYTWAT

SEQ ID NO: 56 - protein, 9 aa, artificial sequence
  synthetic peptide
PGLVYAWST

SEQ ID NO: 57 - protein, 3 aa, artificial sequence
  synthetic peptide
DFY

SEQ ID NO: 58 - protein, 3 aa, artificial sequence
  synthetic peptide
FYE

SEQ ID NO: 59 - protein, 3 aa, artificial sequence
  synthetic peptide
YEE

SEQ ID NO: 60 - protein, 3 aa, artificial sequence
  synthetic peptide
YQE

SEQ ID NO: 61 - protein, 12 aa, artificial sequence
  synthetic peptide
NEEDFYQIQLYE

SEQ ID NO: 62 - protein, 12 aa, artificial sequence
  synthetic peptide
RQIDFYQEIQFK

SEQ ID NO: 63 - protein, 12 aa, artificial sequence
  synthetic peptide
DDFYGQPREQVR

SEQ ID NO: 64 - protein, 3 aa, artificial sequence
  synthetic peptide
YFD

SEQ ID NO: 65 - protein, 3 aa, artificial sequence
  synthetic peptide
EYF

SEQ ID NO: 66 - protein, 3 aa, artificial sequence
  synthetic peptide
EEY

SEQ ID NO: 67 - protein, 3 aa, artificial sequence
  synthetic peptide
EQY

SEQ ID NO: 68 - protein, 12 aa, artificial sequence
  synthetic peptide
EYLQIQYFDEEN

SEQ ID NO: 69 - protein, 12 aa, artificial sequence
  synthetic peptide
KFQIEQYFDIQR

SEQ ID NO: 70 - protein, 12 aa, artificial sequence
  synthetic peptide
RVQERPQGYFDD

SEQ ID NO: 71 - protein, 8 aa, artificial sequence
  synthetic peptide
AAERGLFE

SEQ ID NO: 72 - protein, 7 aa, artificial sequence
  synthetic peptide
AAERGLF

SEQ ID NO: 73 - protein, 6 aa, artificial sequence
  synthetic peptide
AAERGL

SEQ ID NO: 74 - protein, 5 aa, artificial sequence
  synthetic peptide
AAERG

SEQ ID NO: 75 - protein, 4 aa, artificial sequence
  synthetic peptide
AAER

SEQ ID NO: 76 - protein, 3 aa, artificial sequence
  synthetic peptide
AAE

SEQ ID NO: 77 - protein, 2 aa, artificial sequence
  synthetic peptide
AA

SEQ ID NO: 78 - protein, 9 aa, artificial sequence
  synthetic peptide
AAERGLAED

SEQ ID NO: 79 - protein, 9 aa, artificial sequence
  synthetic peptide
AAERGLFAD

SEQ ID NO: 80 - protein, 9 aa, artificial sequence
  synthetic peptide
AAERGLFEA